(19) United States
(12) Patent Application Publication
(10) Pub. No.: US 2016/0215030 A1
(43) Pub. Date: Jul. 28, 2016

(54) Dialysis-Free Process for Aqueous Regenerated Silk Fibroin Solution and Products Thereof

(71) Applicants: Jason Bressner, Lexington, MA (US); Graham Tilburey, St. Louis, MO (US)

(72) Inventors: Jason Bressner, Lexington, MA (US); Graham Tilburey, St. Louis, MO (US)

(21) Appl. No.: 14/608,187

(22) Filed: Jan. 28, 2015

(51) Int. Cl. C07K 14/435

(52) U.S. Cl. CPC C07K 14/43586

(57) ABSTRACT

The present invention provides a dialysis-free process for the generation of aqueous regenerated silk fibroin solutions. A degumming reactor is presented that enables scalable batch degumming. As well, the use of diafiltration and desalting columns are introduced for the purification of silk fibroin solutions, representing a set of techniques that isolate solubilized silk fibroin through the efficient removal of a solubilization agent while implicitly availing the increase in concentration of otherwise dilute aqueous silk fibroin solutions.

### CROSS-REFERENCE TO RELATED APPLICATIONS

The present application claims the priority of U.S. provisional application No. 61/932,247 filed on Jan. 28, 2014 and is included herein as a reference.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The present invention is in the technical field of materials science. More particularly, the present invention is in the technical field of biopolymers, specifically silk.

Silk refers to a natural biomaterial produced as fibers by well over one thousand members of the arthropod phylum, most notably the domesticated silkworm, Bombyx mori. Silk is comprised of fibroin and sericin; fibroin is the main constituent of silk fibers, whereas sericin, comprising approximately 30% of the mass of silk, envelops the fibroin fibers. Notably, sericin is believed to produce an undesirable immunogenic response upon introduction within the human body and thus is viewed as a waste product in the context of implantable products. Therefore, fibroin is customarily isolated by a process known as degumming. This step can be performed via methods that include alkali degumming, enzymatic degumming, plasma degumming, or high pressure degumming.

Fibroin can be converted from solid fibers into a liquid form via solubilization. Previous patents pertaining to the solubilization of silk fibroin date back as far as the mid 1930's, including: Pat. No. 1,966,756 by Gajewski, dated Jul. 17, 1934; Pat. No. 2,006,507 by Mahn, dated Jul. 2, 1935; and Pat. No. 2,010,918 by Fink, dated Aug. 13, 1935. A plurality of solubilization agents, namely salt solutions, can be employed to convert solid silk fibroin into a liquid that is comprised of solubilized silk within a salt solution. Pat. No. 7,751,985 by Li, dated Jul. 6, 2010, and Pat. No. 5,252,285 by Lock, dated Oct. 12, 1993, both elaborate on preferred solutions in which silk fibroin can be solubilized.

Aqueous regenerated silk fibroin is purified by removal of said solubilization agents from the solubilized silk, replacing the solubilization agents with water. In the art, this replacement is conventionally done through static dialysis, whereby ions from the solubilization agent are removed from the silk fibroin solution through the use of a dialysis membrane and a series of water changes. This membrane typically possesses a molecular weight cut off (MWCO) of 3.5 kilodaltons (kDa) or greater. Water is normally used as the dialysate. This process is relatively slow, on the order of days. Therefore, static dialysis techniques represent a bottleneck in terms of commercial-scale production.

Conventional processes for the generation of aqueous silk fibroin solutions are greatly limited in terms of production speed and volume capacity. A standard process, as described by Rockwood (Nature Protocols 6, 1612-1631 (2011)) consumes 4 days. Furthermore, the resulting liquids are too dilute for direct translation into useful products. Kaplan, in Pat. No. 7,635,755, dated Dec. 22, 2009, introduced a method to increase the concentration of silk fibroin solution via dialysis against a hygroscopic polymer.

One alternative to dialysis can be found within the art and is worth noting. Pat. No. 8,309,689 by Yang, dated Nov. 13, 2012 relies on the application of shear stress within a centrifuge to form a precipitate that isolates silk fibroin from its surroundings. This dialysis-free method yields silk fibroin in the solid state, however, rather than in the liquid one.

After decades of relative dormancy, a renewed interest has arisen in recent years to apply regenerated silk fibroin solutions towards the development of devices for biomedical research, owing at least in part to the well-established biocompatibility and biodegradability of silk fibroin fibers, which, as a solid, have functioned as medical sutures for thousands of years. Processes have since been introduced to transform regenerated silk fibroin from a liquid into solid or semi-solid objects such as films, fibers, foams, gels, scaffolds for tissue engineering, microparticles and nanoparticles. Further, a number of these resulting products contain drugs, cells and other molecules to confer added functionality. Selected examples include Pat. No. 8,048,989 by Tsukada, dated Nov. 1, 2011, and Pat. No. 8,071,722 by Kaplan, dated Dec. 6, 2011.

Despite this interest and numerous, albeit nascent, efforts to commercialize technologies built upon silk fibroin, these efforts have been limited by the absence of an efficient and scalable process for the conversion of solid natural silk material, such as cocoons, bave silk, silk waste or silk powder, into a aqueous regenerated silk fibroin solution.

### SUMMARY OF THE INVENTION

The present invention provides a process for the generation of aqueous regenerated silk fibroin solutions. Degumming is performed under fine temperature control while the silk cocoons are agitated, thereby increasing the rate at which sericin is separated from the fibroin. Care is given to optimize the interface between undegummed silk and the degumming solution. Solubilization is performed under physical agitation to optimize the rate at which silk is carried into, solution. Diafiltration techniques, including tangential flow filtration, and desalting columns are presented as purification steps to render an aqueous regenerated silk fibroin solution.

In one embodiment, an aqueous regenerated silk fibroin solution is provided, of a concentration greater than 10 wt %, without the requirement of any explicit concentration step. This silk fibroin solution lends to the immediate creation of products including scaffolds for tissue engineering as well as fibers.

### BRIEF DESCRIPTION OF THE DRAWINGS

FIGS. 1 and 2 depict the relationship between temperature control and time for the degumming step of the invention. The use of an internal proportional-integral-derivative controlled heating element allows for greater temperature stabilization and thus improved process control.

FIG. 3 depicts a full-size, three-dimensional silk scaffold of a human heart. The ability to produce organ-sized scaffolds demonstrates one scale of silk output from the present invention.

### DETAILED DESCRIPTION OF THE INVENTION

The invention described herein is a method to produce an aqueous regenerated silk fibroin solution without the use of dialysis.

Said regenerated silk fibroin solution is produced through the multi-step treatment of solid silk materials, preferably cocoons or their derivatives, including silk fragments, bave silk, waste silk and silk powder. The most common source of silk cocoons is the domesticated silkworm, Bombyx mori. Notably, while spiders also produce silk, they are cannibalistic by nature and do not lend to silk farming, though spider silk can be produced successfully via transgenic means.

The treatment is comprised broadly of three steps: degumming, solubilization, and purification.

Degumming refers to the separation of the outer coating of each silk strand, a layer that is comprised of sericin, from the fibroin which comprises the core of each silk strand. Degumming is performed in a vessel, comprised preferably of stainless steel. Preferably, all or most of the inner surface of the vessel is heated. Throughout this document, the vessel will be referred to as a reactor. The reactor is capable of temperature regulation, using, for example, an internal proportional-integral-derivative controlled heating element, an auxiliary heating element, or some combination thereof.

The reactor is populated with a degumming solution. Degumming solutions include, but are not limited to the following: water pH adjusted with an alkali to pH 10.0 or greater; 7 Molar or greater Urea solution; and 0.01 Molar or greater Sodium Carbonate solution.

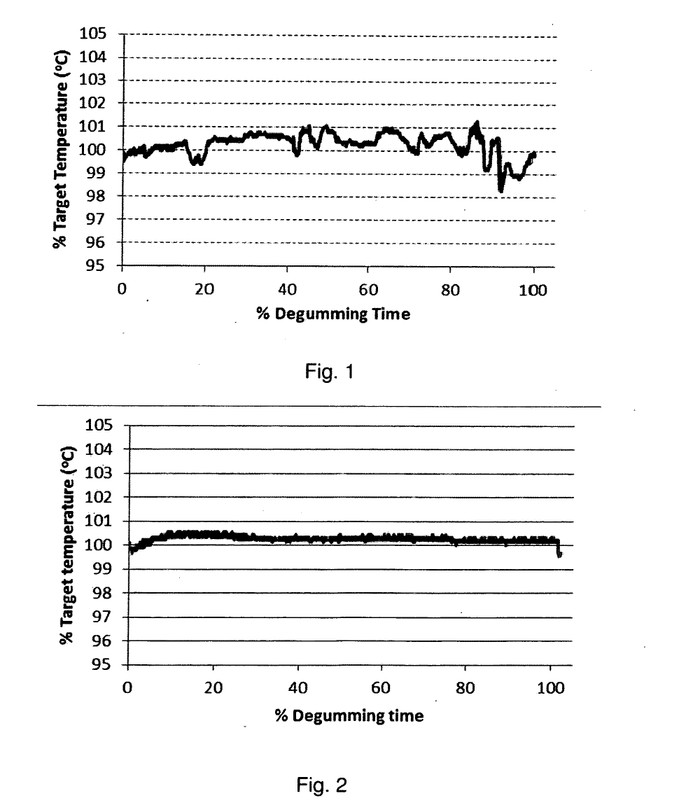

FIGS. 1 and 2 illustrates temperature regulation within the reactor in the presence of a degumming solution. The Graph in FIG. 1 depicts the baseline relationship between temperature and time under conventional degumming, absent temperature regulation. The Graph in FIG. 2 depicts the relationship between temperature and time with the presence of temperature regulation as described in the current invention.

The temperature of the degumming solution is greater than 45° C. and less than 105° C., whereupon solid silk material is introduced for degumming. The degumming process occurs for a time period greater than 3 minutes and less than 300 minutes, depending on the solution temperature, the mass of silk that is introduced and its geometry.

Degumming time is optimized by the use of physical agitation, moving the silk fibroin within the reactor relative to the heated degumming solution, or, preferably, circulating the degumming solution relative to the silk fibroin, As the sericin is removed from the silk fibers, the fibroin can aggregate, making the inner portions of the mass less accessible for further degumming. This is best addressed by harnessing the silk in portions of so that it remains well-distributed throughout the degumming solution, thereby continuously maximizing the exposed surface area of undegummed silk to the degumming solution. In one preferred embodiment, this is accomplished through the use of stainless steel mesh compartments.

Upon removal from the reactor, the fibroin is rinsed in water to remove any residual sericin or degumming solution.

Optionally, degummed silk fibroin can be dried using mechanical means, namely any individual approach or combination of approaches that includes physical pressure such as wringing, spin-drying, the application of heat such as in an oven, and dessication. Alternatively, complete drying can be circumvented by approximating the fibroin mass as 70% of the original silk material mass. The difference in mass between the wet fibroin and the expected mass of the dry fibroin can be attributed to water. Correspondingly, through calculation, the concentration of the solubilization agent can be increased carefully prior to being combined with the wet fibroin; thus, when added to the fibroin and mixed, the concentration of the solubilization agent solution in the presence of the wet silk is diluted to match the desired concentration.

Silk fibroin is solubilized through immersion in one of a selection of solubilization agents. The solubilization agents most commonly used are solutions derived from chaotropic salts, though a number of alternatives can be found in the art. These agents include, but are not limited to the following: aqueous lithium thiocyanate (LiSCN), sodium thiocyanate (NaSCN), calcium thiocyanate (Ca(SCN)₂), magnesium thiocyanate (Mg(SCN)₂), calcium chloride (CaCl₂), lithium chloride (LiCl), lithium bromide (LiBr), zinc chloride (ZnCl₂), magnesium chloride (MgCl₂), copper salts such as copper nitrate (Cu(NO₃)₂), copper ethylene diamine (Cu(NH₂CH₂CH₂NH₂)₂(OH)₂) and Cu(NH₃)₄(OH)₂, Ajisawa's reagent (CaCl₂/ethanol/water), calcium nitrate (Ca(NO₃)₂), sodium iodide (Nap, lithium nitrate (LiNO₃), magnesium nitrate (Mg(NO₃)₂), zinc nitrate (Zn(NO₃)₂).

Heat also is applied to promote the solubilization of fibroin, using an temperature between 40° C. and 80° C. The silk fibroin is fully solubilized within a time period between 15 minutes and 16 hours, depending mainly upon the degumming parameters. Poking or stirring the silk fibroin/solubilizing agent mixture periodically helps to maintain an optimal interface between exposed unsolubilized silk and the solubilization agent. This agitation can accelerate the rate of fibroin solubilization appreciably.

The silk fibroin solution is isolated from the solubilization agent via the final step of the process, purification. This step may be referred to as desalination, in consideration that the vast majority of practitioners employ a salt to solubilize silk fibroin. However, the same procedure is equally applicable when alternate non-salt molecules are utilized.

In the present invention, diafiltration, desalting columns or any combination thereof serve as purification techniques through which a solubilization agent can be removed from the solubilized silk fibroin solution. Tangential flow filtration (TFF) is one technique that applies diafiltration in the removal of the solubilization agent; said agent is eliminated in the filtrate, which is known equivalently as the permeate. In the TFF process, the presence of a concentrating step requires that the original input solution, containing both solubilization agent and solubilized silk fibroin, first be diluted to an acceptable viscosity for suitable input into the TFF system. Passing through the system, the resulting permeate contains water as well as the solubilization agent; the retentate contains an increasingly purified regenerated silk fibroin solution which has been both concentrated and desalted within a single technique.

Alternatively, a desalting column can be used to separate the silk fibroin from the solubilization agent or its ions. The bed height and column diameter are proportional to the volume of solubilized silk fibroin solution to be purified. Before loading the fluid comprised of both solubilization agent and solubilized silk fibroin onto the column, this liquid must be diluted to lower its viscosity. The silk fibroin protein elutes first using water as the mobile phase. The product can be detected, for example, by monitoring absorption at 280 nanometers. Alternatively, a Bradford assay can be used to detect the elution of the silk fibroin protein.

Alternatively, the TFF device can be used in combination with the desalting column, whereby the product eluted from the desalting column is concentrated and further purified using the TFF device.

One added benefit from the use of a desalting column is the ability to separate fragments of silk fibroin on the basis of their molecular weight. This control allows for the creation of silk fibroin solutions possessing tunable mechanical properties.

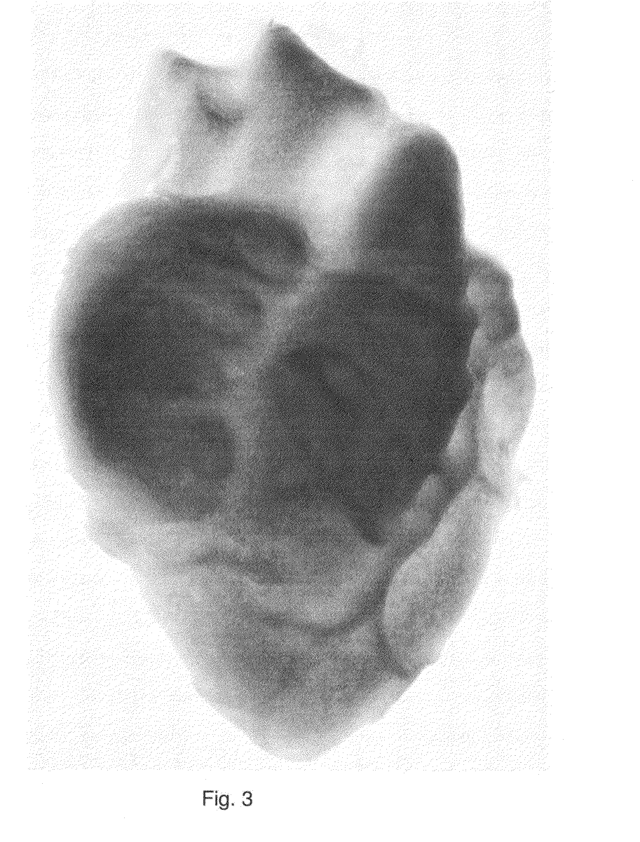

Furthermore, the silk fibroin solutions resulting from this invention can be converted quickly into solid and gel products such as scaffolds for use in tissue engineering. Without the need for any explicit concentration step, silk fibroin solutions can be poured directly onto a dissolvable matrix such as table salt, yielding organ-sized scaffolds (and larger) rapidly, as shown in FIG. 3. A rigid scaffold is formed from a gel intermediate in approximately 24 hours, and the dissolvable matrix can be removed in its entirely immediately thereafter.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

What is claimed is:

1. A dialysis-free method to produce an aqueous regenerated silk fibroin solution, comprising the steps of:
(a) degumming a natural silk material to isolate silk fibroin;
(b) solubilizing silk fibroin in a solubilization agent; and
(c) purifying the solubilized silk fibroin via diafiltration and/or at least one desalting column.

2. The method of claim 1, wherein the natural silk material is derived from domesticated silkworm cocoons.

3. The method of claim 1, wherein the natural silk material is derived from any other arthropod.

4. The method of claim 1, wherein a degumming reactor is comprised of stainless steel.

5. The method of claim 1, wherein the degumming reactor includes an internal proportional-integral-derivative heating element.

6. The method of claim 1, wherein the degumming reactor includes an auxiliary heating element.

7. The method of claim 1, wherein silk in the degumming reactor is housed in a series of chambers to limit aggregation.

8. The method of claim 1, wherein silk fibroin is subject to physical agitation during solubilization.

9. The method of claim 1, wherein a permeate volume removed during diafiltration is replaced by water.

10. The method of claim 1, whose start-to-finish time is 24 hours or less.

11. The method of claim 1, whose start-to-finish time is 12 hours or less.

12. The method of claim 1, whose start-to-finish time is 4 hours or less.

13. The aqueous regenerated silk fibroin solution of claim 1, whereby said solution has a silk fibroin concentration of 5 wt % or greater.

14. The aqueous regenerated silk fibroin solution of claim 1, whereby said solution has a silk fibroin concentration of 10 wt % or greater.

15. The aqueous regenerated silk fibroin solution of claim 1, whereby said solution has a silk fibroin concentration of 15 wt % or greater.

16. The aqueous regenerated silk fibroin solution of claim 1, whereby said solution has a silk fibroin concentration of 20 wt % or greater.

17. The aqueous regenerated silk fibroin solution of claim 1, whereby the conductivity of the regenerated silk fibroin solution is less than 1 siemen.

18. The aqueous regenerated silk fibroin solution of claim 1, whereby the conductivity of the regenerated silk fibroin solution is less than 100 microsiemens.

19. The aqueous regenerated silk fibroin solution of claim 1, whereby the resulting volume is greater than 1 liter.

20. The aqueous regenerated silk fibroin solution of claim 1, whereby the resulting volume is greater than 100 liters.

21-25. (canceled)
